(19) United States
(12) Patent Application Publication
(10) Pub. No.: US 2023/0031279 A1
(43) Pub. Date: Feb. 2, 2023

(54) CONDUCTIVE THERMOPLASTIC ELASTOMER ELECTRODES, AND METHOD OF MANUFACTURING SUCH ELECTRODES

(71) Applicant: MYANT INC., Toronto (CA)

(72) Inventors: Tony CHAHINE, Toronto (CA); Milad ALIZADEH-MEGHRAZI, Toronto (CA); Ladan ESKANDARIAN, Toronto (CA); Amin MAHNAM, Toronto (CA); Bastien MOINEAU, Toronto (CA); Sahar Golmohammadi ROSTAMI, Toronto (CA); Christopher Robin LEIPHART, Toronto (CA)

(21) Appl. No.: 17/789,735

(22) PCT Filed: Dec. 30, 2020
(86) PCT No.: PCT/CA2020/051809

§ 371 Date: Jun. 28, 2022

Related U.S. Application Data
(60) Provisional application No. 62/955,546, filed on Dec. 31, 2019.

(51) Int. Cl. A61N 1/04; D01F 1/09; D04B 21/20; D04B 1/16; D04B 1/22; D04B 1/24

(52) U.S. Cl. CPC A61N 1/0484; D01F 1/09; D04B 21/20; D04B 1/16; D04B 1/22; D04B 1/24; D04B 21/207; A61B 5/256

(57) ABSTRACT

An apparatus and method of manufacturing same is provided. The apparatus comprises a base layer integrated with an article; an electrode mounted adjacent to a conductive layer, both the electrode and conductive layer mounted on the base layer; an active electrode board in electrical communication with the conductive layer and the electrode, the active electrode board configured receive and/or send electrical signals from the electrode. The electrode comprises filaments or filament yarn knitted into a textile. The filaments or filament yarn comprise thermoplastic elastomers (TPE) blended with one or multiple conductive filler/s for improving impedance at the skin-electrode interface.

### FIELD

This relates to textile based electrodes, and in particular to electrodes formed of conductive thermoplastic elastomers.

### BACKGROUND

Electrodes may be used for sensing biopotential signals or imparting electrical stimulation to a person's body. Wet gel has been used in electrodes to reduce impedance at the skin-electrode interface to improve sensing of biopotential signals or the ability to impart electrical energy to a person's body. However, application of a wet gel to a person's body may be difficult or undesirable for certain applications.

### SUMMARY

In one aspect, the disclosure describes an electrode comprising a contact layer comprising a filament, the filament comprising a thermoplastic elastomer.

In an embodiment, the contact layer comprises a conductive filler blended with the thermoplastic elastomer. The contact layer may comprise a second filament comprising a conductive or nonconductive filament.

In an embodiment, the contact layer comprises yarn comprising the first filament.

In an embodiment, the contact layer is backed with a layer of conductive yarn. The conductive yarn may be silver yarn.

Embodiments may include combinations of the above features.

In another aspect, the disclosure describes an apparatus comprising a base layer integrated with an article; an electrode mounted adjacent to a conductive layer, both the electrode and conductive layer mounted on the base layer; an active electrode board in electrical communication with the conductive layer and the electrode, the active electrode board configured receive and/or send electrical signals from the electrode; wherein the electrode comprises filaments or filament yarn, the filaments or filament yarn knitted into a textile, the filaments or filament yarn comprising thermoplastic elastomers (TPE) blended with one or more conductive fillers.

In an embodiment, the apparatus comprises a first overmould of the active electrode board. The apparatus may also comprise a shield mounted over or inside the overmould of the active electrode board. The apparatus may comprise a second overmould over the first overmould and the shield, the second overmould connecting to the base layer.

In an embodiment, the electrode is configured to convert ionic current at a skin-electrode interface to electrical signals when the electrode is coupled to a user. The active electrode board may comprise a preamplifier for increasing signal strength from the electrode when the electrode is coupled to a user.

In an embodiment, the electrode is configured to apply electrical current to a user.

In an embodiment, the article is an article of clothing.

Embodiments may include combinations of the above features.

In another aspect, the disclosure describes a method of manufacturing a filament for an electrode, the method comprising: providing TPE polymer pellets having desired material properties; combining polymer pellets and conductive filler together to form a conductive TPE; extruding and drawing the conductive TPE into a filament; optionally, forming yarn from the filament; and knitting the yarn and/or filament into an electrode.

In an embodiment, the TPE polymer and conductive filler are comprise biocompatible material for forming a biocompatible yarn and/or filament.

Embodiments may include combinations of the above features.

Further details of these and other aspects of the subject matter of this application will be apparent from the detailed description included below and the drawings.

### DESCRIPTION OF THE DRAWINGS

Reference is now made to the accompanying drawings, in which:

FIG. 1 is a prior art example of an electrode-gel-skin interface.

FIG. 2 is an example electrode-skin interface according to the present disclosure.

FIG. 3 is a side cross-sectional view of an example apparatus according to the present disclosure.

FIG. 4A is an overhead view of an example electrode having a raised form factor. FIGS. 4B and 4C illustrate a side perspective view of the example electrode of FIG. 4A showing the raised form factor.

FIG. 5 is a perspective view of an apparatus according to the present disclosure comprising an overmoulded active electrode board.

FIG. 6 is a perspective view of the overmoulded active electrode board of FIG. 5.

FIG. 7A is a cross sectional view of the apparatus of FIG. 5 along the line A-A. FIG. 7B is a cross sectional view of the apparatus of FIG. 5 along the line B-B.

FIG. 8 is a bottom perspective view of the apparatus of FIG. 5.

FIG. 9 is an illustration of an example sequence of making an apparatus according to the present disclosure.

FIG. 10 is a flow chart depicting a method of manufacturing an apparatus according to the present disclosure.

FIG. 11 is a flow chart depicting a method of manufacturing an electrode.

FIG. 12 is an illustration of an example sequence of making an electrode.

FIG. 13 is an view of conductive polymer formed from thermoplastic elastomeric polymer pellets compounded with conductive filler.

FIG. 14 is a perspective view of an example filament comprising thermoplastic elastomeric and conductive filler.

FIGS. 15A and 15B are each enlarged images of an example filament comprising thermoplastic elastomeric and conductive filler.

FIGS. 16A and 16B are each enlarged images of an example patterns of knit filament.

FIGS. 17A, 17B, 17C, and 17D are each side cross-sectional views of example electrodes according to the present disclosure.

### DETAILED DESCRIPTION

The following description relates to a textile-based electrode formed from thermoplastic elastomer material, suitable for sensing bioiopotential signals including Electromyogram (EMG), Electroencephalogram (EEG), Electrocardiogram (ECG), Electrooculogram (EOG), and Electrogastrogram (EGG), as well as applying current/voltage to body for Functional Electrical Stimulation (FES), Transcranial Current Stimulation (TCS), High-Frequency Alternating Current Stimulation, and/or creating a tactile sensation. The description also describes method(s) of manufacturing the thermoplastic elastomer yarns, electrode(s), and apparatus disclosed herein.

The term “substantially” as used herein may be applied to modify any quantitative representation which could permissibly vary without resulting in a change in the basic function to which it is related. The term “proximate” as used herein may refer to the direction or surface that is closest to a contact point between an electrode and skin of a user. The term “flat form factor” as used herein may refer to an electrode wherein a proximate surface of conductive filament or filament yarn is in the same plane material adjacent to the electrode. The term “raised form factor” as used herein may refer to an electrode wherein a proximate surface of conductive filament or filament yarn is in a plane the above material surrounding and adjacent to the electrode.

An electrode acts as a transducer in converting the ionic current in/on the body into electron currents in conductive wires and electronic circuits, and vice versa. Various activities in the body can be measured, such as heart activity with electrocardiography (ECG), muscle activity with electromyography (EMG), brain activity with electroencephalography (EEG), and eye movements with electrooculography (EOG). The electrode-skin interface is the interface that performs this transduction between the electrode and skin while they are in contact. Electrode-skin impedance at the electrode-skin interface may dictate how well the signal will be transduced, and consequently, the signal quality.

A skin's outer layer is the epidermis, consisting of sublayers including the outermost Stratum Corneum. The Stratum Corneum is made of dead keratin-filled corneocytes arranged in a brick-and-mortar pattern. Lipid lamellae exist between the corneocytes, through which conductive ions need to pass in order to travel from the deeper layer of skin to the electrodes during bipotential measurements. When temperature and humidity increase in the Stratum Corneum, the fluidity of these lipids increases, allowing water and small molecules to move through the lipid lamellae more easily. Thus, hydrating the Stratum Corneum allows for higher signal quality in biopotential measurement.

An electrode may generally be defined as conductive material through which electricity passes to a body of a user and/or is received from the body of a user. A sensor is a subset of electrodes which receives electrical energy for measurement/recordation. In an embodiment, electrode 100 may be used as a sensor. In another embodiment, electrode 100 may be an actuator to inject electrical current/voltage to the body, e.g. for FES to inject electrical pulses to activate muscles.

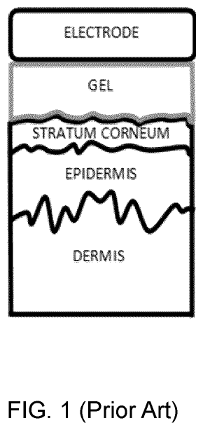

Standard electrodes may use an electrolytic gel to maintain good electrical contact with the Stratum Corneum, creating an ionic path between the electrode and the skin below the Stratum Corneum via conductive ions in the gel. This reduces the skin impedance and allows for improved signal acquisition. The skin-electrode interface for wet electrodes is shown in FIG. 1. However, the standard wet gel electrode used currently, e.g. in healthcare, may have limitations. The adhesive can cause skin irritation and becomes uncomfortable over time, the gel dehydrates with time thus degrading signal quality, and the electrode can be uncomfortable to the user, due to its metallic piece, therefore a soft, textile form is an inconspicuous alternative for continuous health monitoring.

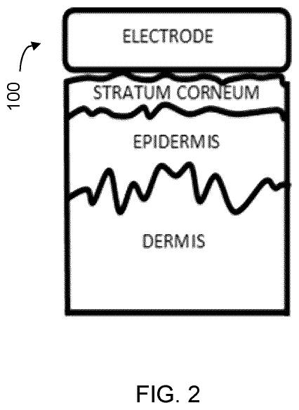

Dry contact electrodes can be categorized according to form factor into textile electrodes, flexible film electrodes, bulk electrodes, pin-shaped electrodes, and microneedles. An example skin-electrode interface for dry electrodes is depicted in FIG. 2, differing in that it does not contain the gel layer. Dry electrodes may be biocompatible, easy to use, comfortable, breathable, lightweight, flexible, washable, durable, and able to maintain good signal quality during electrophysiology testing while at rest and moving. Additionally, textile-based electrodes may be worn on various body parts by attaching them to, or integrating them into, different articles of clothing such as waistbands, sleeves, pants, and headbands.

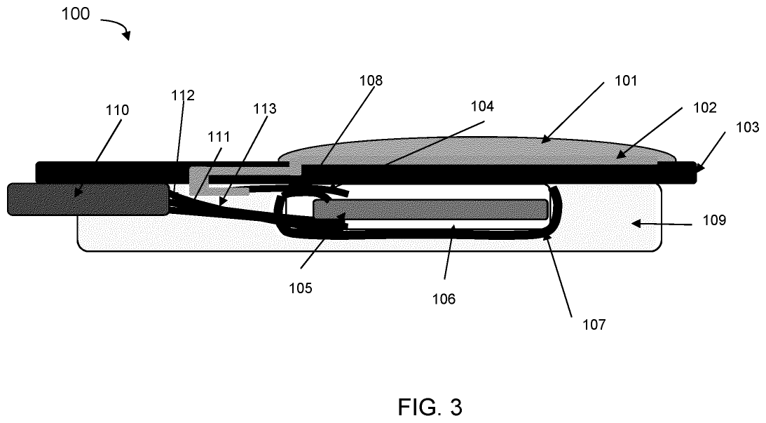

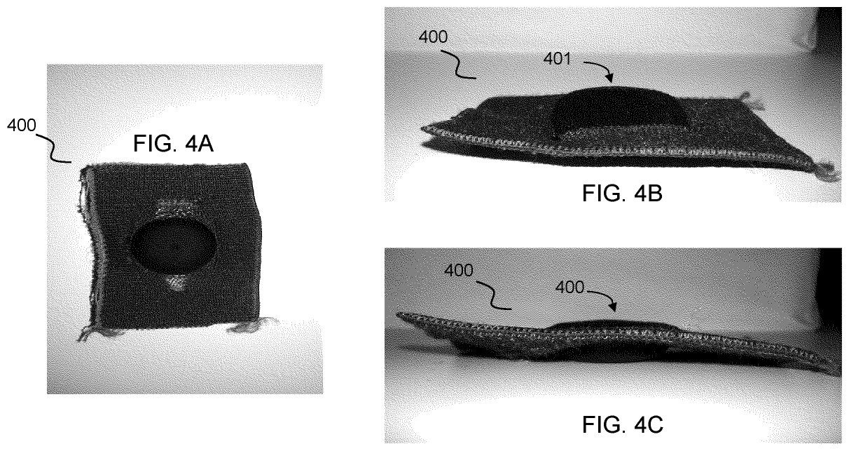

An example developed electrode is illustrated in FIG. 4A-C. The example electrode 400 of FIGS. 4A-C has a raised form factor F (FIG. 3): a circular 2-cm diameter electrode that is knit using conductive Thermoplastic Elastomer filament connected to a more conductive layer on its back. The conductive back layer may be silver-plated nylon, polyester, Kevlar yarn, stainless steel and/or metallic microwire wrapped around polyester, nylon, viscose, rayon, and/or aramid yarn. Conductive layer may be defined beneath contact layer. Contact layer and conductive layer may be mounted on a base layer or be part of an article. In an embodiment, base layer may be a non-conductive double layer fabric. As shown in FIGS. 3, contact layer may have a raised form factor such that contact layer defines a surface in a plane above (i.e. more proximate to a user than) base layer. A raised form factor may promote sustained contact between contact layer and a user. Base layer may be part of an article, e.g. clothing or furniture, having a surface area.

FIGS. 4B and 4C illustrate a side perspective view of the example electrode showing raised form factor 401. Raised form factor 401 may promote sustained contact a surface-to-skin interface between a user and a contact layer of the raised form factor 401.

A plurality of electrodes according to the present disclosure may be part of a garment, and each of the plurality of electrodes may be integral to an article (e.g. clothing) forming a uniform structure.

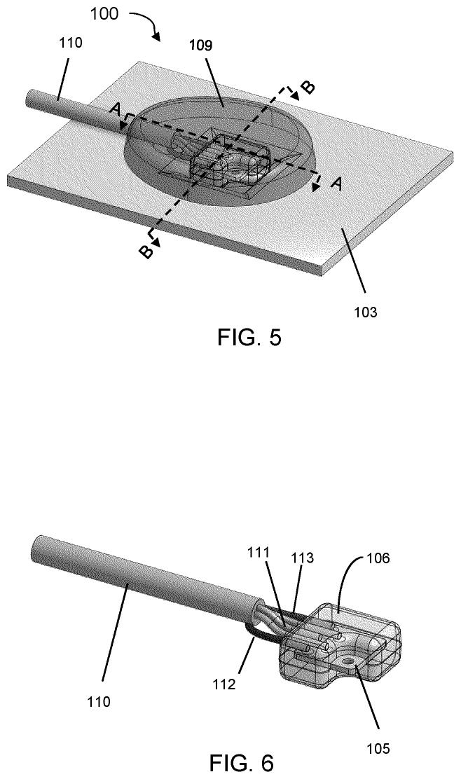

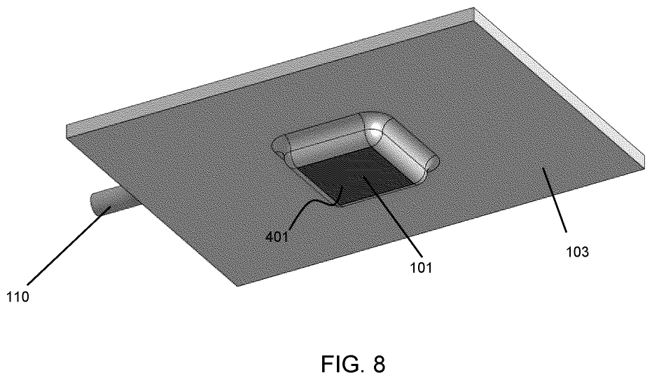

An example apparatus 100 according to the disclosure herein is illustrated in FIGS. 3 and 5. Apparatus 100 is a dry electrode comprising Thermoplastic Elastomers (TPE) combined with conductive material. For example, the conductive material may be a conductive filler (i.e. conductive dopant) or a conductive coating. As illustrated in FIG. 3, contact layer 101 comprises conductive TPE yarn comprising a TPE combined with conductive filler. In another embodiment, contact layer 101 may comprise conductive material coated onto a TPE. Contact layer 101 is in electrical communication with conductive layer 102. In an embodiment, conductive layer 102 may be silver-plated nylon, polyester, Kevlar yarn and/or silver-plated copper microwire wrapped around polyester or nylon yarn 102. Conductive layer 102 may be defined beneath contact layer 101. Contact layer 101 and conductive layer 102 may be mounted on base layer 103 as shown in FIG. 3. In an embodiment, base layer 103 may be a non-conductive double layer fabric. As shown in FIGS. 3, 7A, 7B, and 8 contact layer 101 may have a raised form factor such that contact layer 101 defines a surface in a plane above (i.e. more proximate to a user than) base layer 103. A raised form factor may promote sustained contact between contact layer 101 and a user. Base layer 103 may be part of an article, e.g. clothing or furniture, having a surface area. In an embodiment, the article may comprise a plurality of apparatus 100 which may be integral with the article.

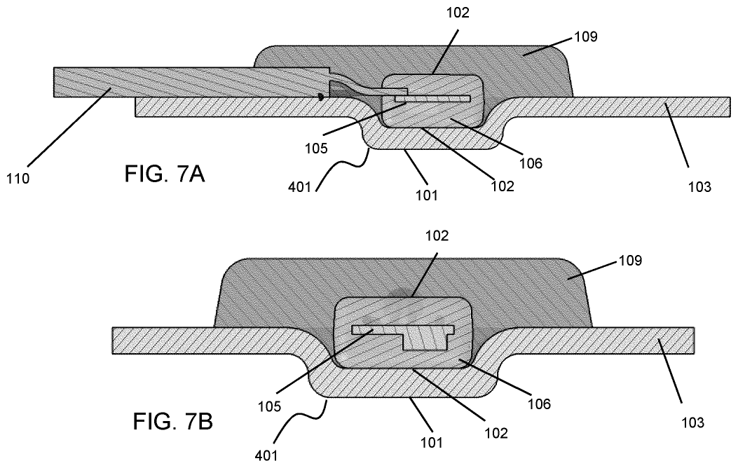

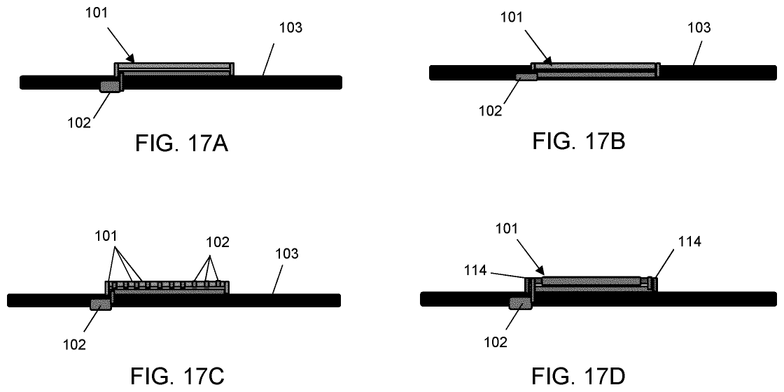

The arrangement of contact layer 101, conductive layer 102, and base layer 103 is not limited to the embodiment illustrated in FIGS. 3, 7A, and 7B. FIG. 17A illustrates an example electrode having a raised form factor, the electrode having a contact layer 101 of fabric knit or woven with TPE yarns; a conductive layer 102 comprising a layer of fabric behind it knit or woven with other conductive yarns, e.g., silver yarns; and a base layer 103 comprising non-conductive yarns. Conductive layer 102 may be connected to a patch of conductive fabric on base layer 103 for connecting the contact layer 101 to electronics. FIG. 17B illustrates another example textile electrode having a flat form factor with the same arrangement of contact layer 101, conductive layer 102 and base layer 103 as in FIG. 17B. FIG. 17C illustrates another example of textile electrode wherein contact layer 101 comprises both conductive TPE yarns and other conductive yarns, which as illustrated may be the same conductive yarn(s) as conductive layer 102 but may also be another type of conductive yarn. The structure of FIG. 17C creates a mixture of TPE and conductive non-TPE yarns in contact with skin of a user. FIG. 17D illustrates another example textile electrode wherein contact layer 101 comprises nonconductive TPE yarns 114 and non-TPE conductive yarn 102 that acts as the electrode.

Conductive layer 102 may be in electrical communication with active electrode board 105 via connection 104. Connection 104 may extend from conductive layer 102 through base layer 103 to active electrode board 105 to provide raw signal input from the contact layer 101/conductive layer 102 to active electrode board 105. Connection 104 may be a wire, an extension of conductive layer 102 directly soldered or crimped to active electrode board 105, a rivet, or other connection member coupling conductive layer 102 to active electrode board 105. Active electrode board 105 may comprise a printed circuit board (PCB) and electronic circuitry. Active electrode board 105 may also comprise a pre-amplifier, or communicate with a pre-amplifier, to record signal(s) with no attenuation from contact layer 101. Pre-amplification may improve signal-to-noise ratio from contact layer 101 as contact layer 101 is a dry electrode having increased impedance at the skin-electrode interface. Integration of active electrode board 105 places pre-amplifier in immediate proximity to the skin-electrode interface at contact layer 101 which may provide improved signal-to-noise ratio. Pre-amplified signal output 111, power input (e.g. Voltage Common Collector) 112, and ground wire 113 may each connect active electrode board 105 with co-axial cable 110 for communication with at least one of a computer, power source, and ground. In an embodiment, at least one of cable 110 and ground wire 113, may be made out of similar material as conductive layer 102, e.g. silver-plated nylon, polyester, Kevlar yarn and/or silver-plated copper microwire wrapped around polyester or nylon yarn.

In an embodiment, active electrode board 105 may be overmoulded with a non-conductive material to form overmould 106 as illustrated in FIGS. 3, 6, 7A, and 7B. Overmould 106 may be a non-conductive TPE. In an embodiment, overmould 106 is created using Technomelt™. Technomelt™ is a polyamide material with a low melting temperature e.g. about 110° C. Technomelt™ can be used in low pressure molding processes, making it compatible for use with active electrode board 105 as the temperature and pressure are low enough to not damage the components on the active electrode board 105 or affect the quality of their soldered joints. Overmould 106 may cover active electrode board 105 with about 1.0 to 1.5 mm coating of material. Wires connecting to the active electrode board 105 may protrude through overmoulding 106. Overmoulding 106 may waterproof the active electrode board 105 and provide strain relief for solder wire connections to a PC board of the active electrode board 105.

An electromagnetic shield 107 may be provided, the shield 107 defining overmould 106 and/or active electrode board 105 within an internal volume of the shield 107. In an embodiment, shield 107 may be made of sheet of silver or other conductive material such as conductive textile sheet(s). Shield 107 may be connected to active electrode board 105 by connection 108. Shield 107 may reduce electromagnetic interference between active electrode board 105 and contact layer 101.

In an embodiment, shield 107 may be formed as a portion of the PCB of active electrode board 105. In this embodiment, the PCB of active electrode board 105 may be a flexible PCB (e.g., made from a flexible and resilient material). In an embodiment, wings are configured to provide an electromagnetic barrier to reduce noise at the contact layer 101 to skin interface. The ground plane of active electrode board 105 may extend along the wings, and may form shield 107 by extending through overmould 106 to wrap around overmould 106.

Overmould 109 may be a non-conductive material. Overmould 109 may be formed from an overmoulded on base layer 103, shield 107, and co-axial cable 110 as shown on FIGS. 3, 5, 7A, and 7B. Overmould 109 may be a non-conductive TPE. Overmould 109 may provide an attachment to base layer 103, e.g. a textile, to secure overmould 106 and active electrode board 105 therein adjacent to base layer 103. Overmould 106 may not be fixed to base layer 103 and permitted to float with respect to base layer 103. In an embodiment, overmould 109 is a TPE having resilient and flexible properties, and overmould 109 may be configured to stretch when (textile) base layer 103 moves.

To join the overmould 106, active electrode board 105, and connection 104 to base layer 103, the overmould 106, active electrode board 105, and connection 104 are positioned in a mold and are then overmoulded with a flexible elastomer material to form overmould 109. The elastomer material may adhere to a knit structure of base layer 103 creating an inseparable assembly which is waterproof. The elastomer material may be a thermoplastic elastomer (TPE) or a two part silicone epoxy. The processing temperature of the TPE may be low enough to not damage base layer 103, e.g. a knit textile. For example, the TPE may be applied at a temperature lower than the melting temperature of the base layer 103. Adhesion between overmould 109 and base layer 103 may be mostly mechanical in nature. To encourage adhesion of the overmould 109 to base layer 103 a few methods may be used. For example, 1) base layer 103, e.g. a knit textile, could be pre-stretched to open up the knit structure allowing the TPE to flow into the yarns creating a mechanical interlock; 2) base layer 103 may be knit to have an open structure which allows the elastomer to flow into the yarns creating a mechanical interlock; 3) materials (i.e. yarns, coating) can be used in the base layer 103, which may have a knit structure that have a melting point similar to overmould 109, which allows some of the material of base layer 103 and the material of overmould 109 to melt together and then solidify as a mix.

Contact layer 101 may comprise TPE(s). A variety of thermoplastic elastomer materials may be used in contact layer 101. Conductive filler may also be blended with the TPE material of contact layer 101 to impart desired conductivity properties. TPE polymer may be provided in pellets/granules which may be melted into molten form and mixed with conductive carbon based fillers to form a homogenous mixture. The homogeneous mixture comprising blended TPE(s) and conductive filler may be formed into a filament yarn. In an embodiment, an extruded filament yarn is formed from the blended TPE and conductive filler. Extruded filament may be knitted as contact layer 101 of electrode 100 having a desired knit pattern suitable for biosensing. U.S. Patent Publication No. US2011/0200821 describes an example system and process that may be used for manufacturing conductive filament yarn, the entire disclosure of which is hereby incorporated by reference herein.

TPE materials for contact layer 101 may be neat polymer matrix materials belonging to two groups: (1) Styrenic Block copolymers, and (2) Polyolefin-Based Elastomers. TPE material may also be thermoplastic Polyurethane Elastomers (TPU), Thermoplastic Polyether Ester/Polyamide elastomer, and silicone base materials such as Polydimethylsiloxane (PDMS).

Conductive filler may be an electrically conductive material and may form 0.5 wt % to 40 wt % of contact layer 101. In an embodiment, conductive filler may be carbon black particles. Carbon-based materials such as carbon nanotubes, graphene, carbon black, acetylene black, and mixture thereof may also be as conductive filler. Conductive filler is not limited to carbon material, and may be metallic nano-fillers such as silver, gold or brass. Conductive fillers may be selected based on (1) Biocompatibility of the conductive filler, (2) Size and morphology, (3) Surface area, (4) Percolation rate, (5) Conductivity, (6) Spinnability.

In an embodiment, contact layer 101 may comprise a filament (or filament yarn) comprising TPE(s) and conductive filler that may have at least one of a Monofilament Diameter of less than or equal to 0.5 mm, more preferably 0.1-0.4 mm; Elastic limit (recoverable stretch) of 100% min, preferably greater than 150%; Filament Conductivity of 1-100 KOhm/m; and Rheology—as measured, e.g., by viscosity and melt flow index, suitable for forming filament yarn when melted.

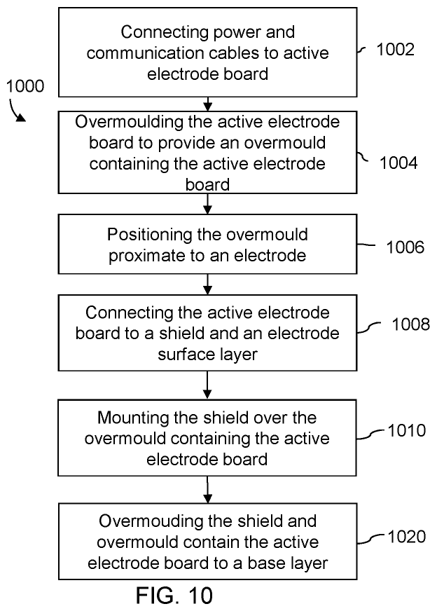

FIG. 10 is a flow chart depicting an example process 1100 for manufacturing an apparatus according to the present disclosure.

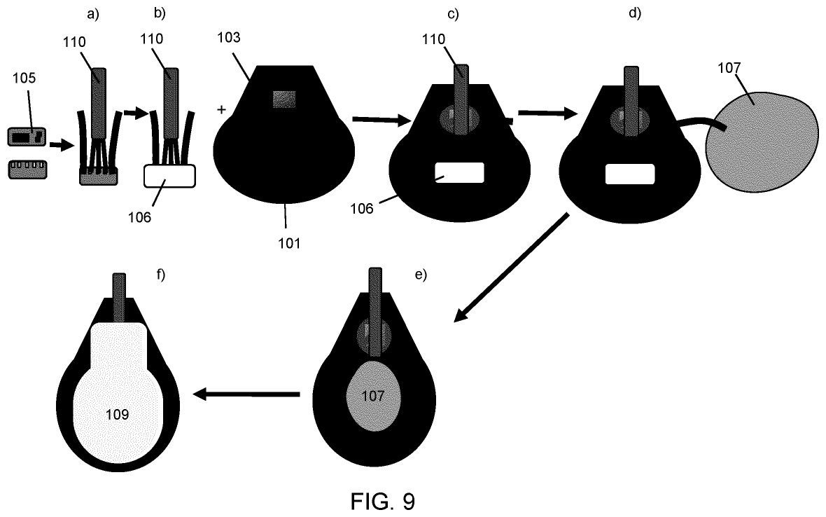

At block 1002, power and communication cables are connected to an active electrode board as illustrated at FIG. 9 a)

At block 1004, the active electrode board is overmoulded to provide an overmould containing the active electrode board as illustrated at FIG. 9 b).

At block 1006, the overmould is positioned proximate to an electrode contact layer. For example overmould 106 may be positioned on an opposing side of an electrode's contact layer 101 as illustrated in FIG. 9 c).

At block 1008, the active electrode board is connected to a shield and electrode contact layer as illustrated in FIG. 9 d).

At block 1010, the shield is mounted over the overmould as illustrated in FIG. 9 e).

At block 1020, the shield and overmould containing the active electrode board are overmoulded to a base layer as illustrated in FIG. 9 f).

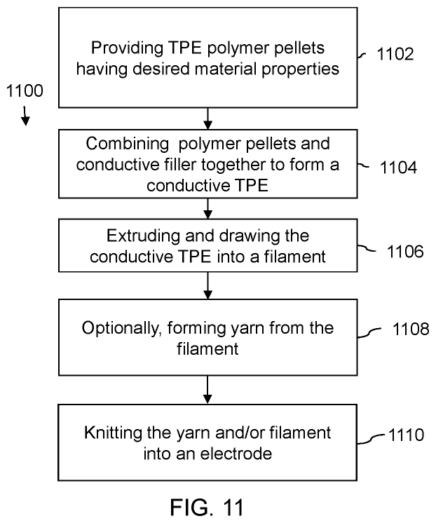

FIG. 11 is a flow chart depicting an example process 1100 for manufacturing an electrode. At block 1102, TPE polymer pellets having desired thermoplastic elastomer material properties are provided.

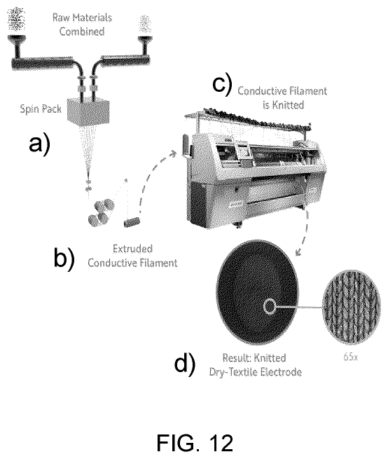

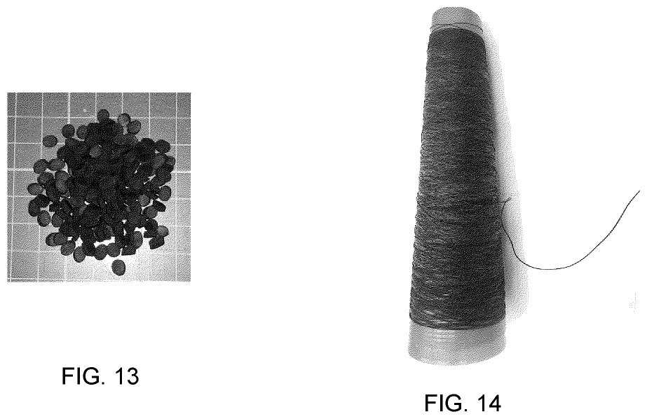

At block 1104, polymer pellets and conductive filler are combined together to form a conductive TPE. In an example, the TPE polymer pellets and conductive filler may be compounded to mix the conductive fillers with TPE polymer forming conductive polymer pellets shown in FIG. 13. The conductive polymer pellets may be added to a hopper for of a melt spinning machine as shown in FIG. 12 a). In an embodiment, the melt spinning machine can be mono-, bi-, tri-, quad-components. Depending on the desired properties of the filament and/or filament yarn, the composition of the yarn may be selected from different components. Each component may have a separate hopper/feeder and heating zone to melt each of the components together—including the conductive polymer pellets which is melted to form conductive TPE. In an embodiment, the TPE polymer pellets and conductive filler may be melted at a temperature from 130 C to 300 C. In another embodiment, the TPE polymer pellet and conductive filler may be melted together at a temperature below 130 C. Components of the filament yarn may include conductive polymer (such as conductive TPE), self-healing materials, far infrared (FIR) particles and microcapsules of phase-change materials for thermal regulation.

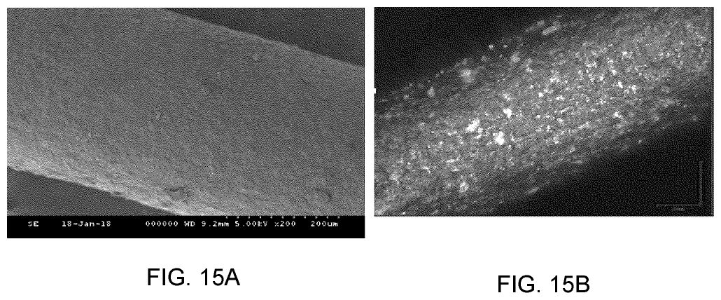

At block 1106, the conductive TPE may be extruded and drawn into a filament. Continuing the above example, as illustrated in FIG. 12 a), the conductive TPE may be extruded and drawn in the melt spinning machine to form filament(s), which may be combined with filaments of other components at a spinneret (shown in FIG. 12 a) to form a filament yarn as discussed at block 1108, which may then be directly solidified by cooling. Different filament structures may be created, such as monocomponent with different diameters (e.g. 50 microns to 400 microns) and bicomponent structures (e.g. core-sheath, lobal, side-by-side, segmented, and Islands-in-the-sea). FIGS. 15A and 15B illustrate microscopic images of extruded mono-filament (single component) comprising conductive TPE.

In an example, the spinneret can be configured to provide a different cross sectional shapes and diameters of extruded filaments. In an embodiment, diameter of the spinneret can be between 50 micron to 1 mm. The extruded filament may be drawn to improve the crystallinity and create thinner filaments. In an embodiment, the diameter of the extruded filament may be drawn to have a diameter in the range of 100 to 500 micron. In an example, the properties (e.g. spinnability, biocompatibility, and conductivity) of a monocomponent filament structure may solely depend on the type of TPE matrix and fillers used.

Filaments according to the present disclosure may have different bi-component structures. Sometimes conductive pellets may not have sufficient mechanical strength to be extruded and drawn only by themselves; accordingly, another polymer may be used as a core material and the conductive polymer of the conductive pellets (which is made through compounding) may be a sheath. Filaments may have various structures such as hollow-fibers or a structures formed from polymer filaments extruded together in multi-component melt-spinning. The filaments may have various cross-section such as, for example, side-by-side, core and sheath, hollow, c-shape, trilobal, islands in the sea, and the like. In an example, an extruded filament may comprise water soluble polymer(s) (e.g. Poly(vinyl alcohol); “PVA”) which may be placed in a water bath after extrusion to remove the water soluble polymer(s). In another example, air may be blown during spinning to create hollow fibers where the sheath is formed from conductive polymer.

At block 1108, optionally filament yarn is formed from the filament(s). Yarn may be formed by a melt spinning machine illustrated at FIG. 12 a). In an embodiment, after extruding the filament, the filament may be wrapped by water soluble polymer(s), e.g. PVA to make a yarn. In an example, mono-component filament comprising conductive TPE may be a core wrapped by water soluble yarns (e.g. PVA) to make the yarn formed from the filament easier to knit. In an embodiment, extruded filament may be coated with powder for better knittability. In an embodiment, the powder is Talc powder. As shown in FIG. 12 b), the filament(s) or filament yarn may be further extruded to a desired dimension. FIG. 14 illustrates an example filament yarn comprising conductive TPE produced according to the disclosure herein.

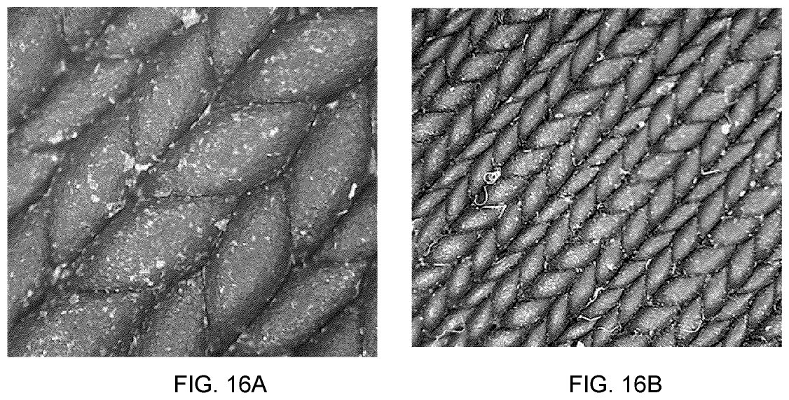

At block 1110, the yarn and/or filament is knit into an electrode. In an example, flat-bed knitting machines illustrated in FIG. 12 c) may knit yarn or filament comprising conductive TPE into electrodes (shown in FIG. 12 d)) having a desired geometry and pattern. Based on the diameter of yarn the gauge of the knitting machine can be chosen. The thinner the yarn or filament, the higher the gauge of the machine to increase the resolution of the knitted electrode. In an example, different structures of electrode may be knit such as an electrode having a raised form factor or a flat form factor. The electrode may be made with various other textile manufacturing processes such as jacquard weaving, circular jacquard knitting, warp knitting and embroidery based on the desired properties of the electrode. For example jacquard weaving may be used to provide structures having improved dimensional stability; circular knitting may provide a structure with improved flexibility that may be produced quickly; and warp knitting may provide different yarn diagonals into knitted structures. Flat bed knitting may be used to apply different functionalities into the electrode, such as by inserting a functional laminate, RFID, and/or pH/sweat/moisture sensor behind the electrode during the manufacturing process. The size of the electrode, its geometry (e.g. square, oval, circular) may also be selected to improve performance for example by minimizing impedance at the skin-electrode interface. Yarn and/or filament may be knit into a desired pattern. Example patterns of knit filament are shown in FIGS. 16A and 16B which are enlarged views of a knit pattern under 225× and 15-65× magnification respectively. The size, shape and materials of the yarn may be selected to enhance the performance of the electrode for receiving and/or recording a specific type of signal based on its frequency and amplitude range. In an example, an electrode comprises conductive thermoplastic elastomer, and the amount of conductive fillers, type of conductive filler, and structure of the yarn or filament such as diameter, elongation, tensile strength, cross-section, and geometrical structure may be varied to suit a desired application e.g. ECG, EMG, EEG, FES, etc.

An electrode according to the disclose herein may be used for different applications such that similar filament can be used in electrodes for bio-signal monitoring, functional electrical stimulation, heat generation, motion sensing, moisture sensing, respiration sensing, etc. Further, a single strand of the extruded filament may be knitted as an electrode such that material consumption may be reduced compared with other conductive filaments, e.g. carbon-contained nylon, silver plated nylon, etc. In some examples, because an extruded filament according to the disclosure herein may comprise silicone and/or rubber, an electrode made from the extruded element may have more grip when in contact with skin which may decrease motion artifact and retrieve bio-signals with higher resolution. In another example, electrodes according to the disclosure herein are biocompatible and such that they may be in contact with a human body for long-term monitoring and medical applications.

The electrode and/or conductive TPE disclosed herein may also be used for strain gauge. In an embodiment, the resistance of a filament according to the disclose herein may change by stretching, causing the distance between conductive particles in filament matrix to change; in turn, causing resistance to change. By measuring the change in resistance as the electrode, the electrode and/or filament may be used for stretch/motion sensing.

The electrode and/or conductive TPE disclosed herein may also be used for in heat applications. In an example, the conductive fillers, e.g. carbon-based fillers, may create high resistance so filament formed from conductive TPE polymer, or a sheet of the conductive TPE polymer, it can be used as a heating element by running an electric current through it. High conductivity yarns/filaments may be used as a bus and the extruded filament/sheet as heating element—due to the high resistance of sheet/filament, it will heat up and can be used in heat applications.

The electrode and conductive TPE disclosed herein may also be used as a moisture sensor. The polymer matrix may be selected such that it's sensitive to a group of solvents and it swells once it comes in contact with those types of solvents/solutions therefore the distance between its conductive particles will change so its resistance will change and it can be sensitive to moisture.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

The claims are not intended to include, and should not be interpreted to include, means-plus- or step-plus-function limitations, unless such a limitation is explicitly recited in a given claim using the phrase(s) “means for” or “step for,” respectively.

What is claimed is:

1. An electrode comprising:
a contact layer comprising a first filament, the filament comprising a thermoplastic elastomer.

2. The electrode of claim 1, wherein the contact layer comprises a conductive filler blended with the thermoplastic elastomer.

3. The electrode of claim 2, wherein the contact layer comprises a second filament comprising a conductive or nonconductive filament.

4. The electrode of any claim 1, wherein the contact layer comprises yarn comprising the first filament.

5. The electrode of claim 1, wherein the contact layer is backed with a layer of conductive yarn.

6. The electrode of claim 5, wherein the conductive yarn is silver yarn.

7. An apparatus comprising:
a base layer integrated with an article;
an electrode mounted adjacent to a conductive layer, both the electrode and conductive layer mounted on the base layer;
an active electrode board in electrical communication with the conductive layer and the electrode, the active electrode board configured receive and/or send electrical signals from the electrode;
wherein the electrode comprises filaments or filament yarn, the filaments or filament yarn knitted into a textile, the filaments or filament yarn comprising thermoplastic elastomers (TPE) blended with one or more conductive fillers.

8. The apparatus of claim 7, comprising a first overmould of the active electrode board.

9. The apparatus of claim 8, comprising a shield mounted over or inside the overmould of the active electrode board.

10. The apparatus of claim 9, comprising a second overmould over the first overmould and the shield, the second overmould connecting to the base layer.

11. The apparatus of claim 7, wherein the electrode is configured to convert ionic current at a skin-electrode interface to electrical signals when the electrode is coupled to a user.

12. The apparatus of claim 11, wherein the active electrode board comprises a preamplifier for increasing signal strength from the electrode when the electrode is coupled to a user.

13. The apparatus of claim 7, wherein the electrode is configured to apply electrical current to a user.

14. The apparatus of claim 7, wherein the article is an article of clothing.

15. A method of manufacturing a filament for an electrode, the method comprising:
providing TPE polymer pellets having desired material properties;
combining polymer pellets and conductive filler together to form a conductive TPE;
extruding and drawing the conductive TPE into a filament;
optionally, forming yarn from the filament; and
knitting the yarn and/or filament into an electrode.

16. The method of claim 15, wherein the TPE polymer and conductive filler are comprise biocompatible material for forming a biocompatible yarn and/or filament.
